(19) United States
(12) Patent Application Publication
(10) Pub. No.: US 2016/0143869 A1
(43) Pub. Date: May 26, 2016

(54) STABLE BROMFENAC SOLUTION

(71) Applicants: Mandar V. SHAH, Haryana (IN); SENTISS RESEARCH CENTER, Haryana (IN)

(72) Inventors: Mandar V. SHAH, Haryana (IN); Prafulla CHAUDHARI, Haryana (IN); Deepak BAHRI, Haryana (IN)

(73) Assignee: SENTISS RESEARCH CENTER, Haryana (IN)

(21) Appl. No.: 14/898,268

(22) PCT Filed: Jun. 17, 2014
(86) PCT No.: PCT/US2014/042724

§ 371 Date: Dec. 14, 2015

(30) Foreign Application Priority Data
Jun. 19, 2013 (IN) 1816/DEL/2013

(51) Int. Cl. A61K 31/196; A61K 47/02; A61K 47/18; A61K 47/26; A61K 9/00; A61K 47/12

(52) U.S. Cl. CPC A61K 31/196; A61K 9/0048; A61K 47/12; A61K 47/183; A61K 47/26; A61K 47/02

(57) ABSTRACT

The present invention provides a stable, aqueous solution comprising bromfenac or a pharmacologically acceptable salt, polymorph, ester or hydrate thereof and/or pharmaceutically acceptable excipients wherein the invention is preferably devoid of an alkyl aryl polyether alcohol type polymer such as tyloxapol, a polyethylene glycol fatty acid ester such as polyethylene glycol monostearate and BAC. Also the present invention is preferably devoid of antioxidants such as sulfite but not limited to sodium sulfite, potassium sulfite and the like. The present invention also provides for a method for treating ocular inflammation and pain, e.g., after cataract surgery, wherein the method comprises topical application of a formulation according to the present invention to the eye of a patient in need thereof.

### CROSS-REFERENCE TO RELATED APPLICATION

This application claims priority to IN provisional application No. 1816/DEL/2013 filed Jun. 19, 2013, the contents of which are incorporated herein by reference in their entirety.

### FIELD OF THE INVENTION

The present invention is directed to stable, aqueous solution comprising a non-steroidal anti-inflammatory drug (NSAID) for the treatment of ocular inflammation and pain after cataract surgery.

Specifically, the present invention is directed to a stable, aqueous solution comprising bromfenac or a pharmacologically acceptable salt, polymorph, ester or a hydrate thereof and/or pharmaceutically acceptable excipients for the treatment of ocular inflammation and pain after cataract surgery. The present invention is preferably devoid of stabilizers and antioxidants such as sulfite(s) preferably sodium sulphite and potassium sulfite.

Further, the solution of the present invention can be formulated both as multi-dose as well as unit dose composition.

The present invention provides a method for treating ocular inflammation and pain after cataract surgery wherein the method comprises a once a day topical application to the eye of the patient in need of a antioxidants-free, stable, aqueous solution comprising bromfenac or a pharmacologically acceptable salt, polymorph, ester or a hydrate thereof and/or pharmaceutically acceptable excipients wherein more specifically, the present invention is devoid of benzalkonium chloride (BAC).

### BACKGROUND

Bromfenac is 2-amino-3-(4-bromobenzoyl)phenylacetic acid (Japanese patent no. 2683676 corresponding to U.S. Pat. No. 4,910,225). Bromfenac has been practically used as its sodium salt in the form of eye drops in the field of ophthalmology.

Bromfenac (chemical name 2-amino-3-(4-bromobenzoyl)phenylacetic acid) is a non-steroidal anti-inflammatory agent, is disclosed in JP-A-23052/1977 and its corresponding U.S. Pat. No. 4,045,576. Bromfenac is effective against inflammatory diseases (e.g. blepharitis, conjunctivitis, scleritis, postoperative inflammation) of the extraocular segment or the anterior ocular segment in the field of ophthalmology, and in particular, its efficacy for treating uveitis is equal to nonsteroidal anti-inflammatory agents which have previously been used in the field of ophthalmology, and its sodium salt has been practically used in the form of eye drops. The eye drop as mentioned above is designed to stabilize 2-amino-3-(4-bromobenzoyl)phenylacetic acid by means of addition of a water-soluble polymer (e.g. polyvinylpyrrolidone, polyvinyl alcohol, etc.) and a sulfite (e.g. sodium sulfite, potassium sulfite, etc.) (Japanese patent No. 2,683,676 and its corresponding U.S. Pat. No. 4,910,225).

In addition, as an eye drop other than the above-mentioned one, Japanese patent No. 2,954,356 (corresponding to U.S. Pat. Nos. 5,603,929 and 5,653,972) discloses a stable ophthalmic composition which comprises incorporating an antibacterial quaternary ammonium polymer and boric acid into an acidic ophthalmic agent. The acidic agent described therein includes, for example, 2-amino-3-(4-bromobenzoyl)phenylacetic acid.

Further, in Japanese patent No. 2,954,356, there is the following description—“Benzalkonium chloride is a widely used preservative in ophthalmic solutions. However, benzalkonium chloride and other quaternary ammonium compounds are generally considered to be incompatible with ophthalmic compositions of drugs with acidic groups, such as non-steroidal anti-inflammatory drugs. These preservatives lose their ability to function as they form complexes with the charged drug compounds”.

It is well known in the prior that small organic compounds, such as benzalkonium chloride (BAC), chlorhexidine, thimerosal have excellent antimicrobial activity; however, it is now known that these small organic antimicrobials are often toxic to the sensitive tissues of the eye and can accumulate in cornea, contact lenses, particularly soft, hydrophilic contact lenses. Medications with BAC may cause disruption of the corneal surface with lower concentrations of BAC.

Gasset and Grant et al. showed that BAC accumulates in ocular tissue and remains there for long periods, adversely affecting both the corneal surface and the conjunctiva. Therefore, cessation of the medications may not immediately improve the condition and function of the ocular surface. These findings also suggest that corneal cell necrosis may occur in some patients who are taking multiple BAC-preserved ocular medications over long periods of time, even when the amount of BAC in any one medication is below the threshold concentration at which necrosis occurs. When bromfenac eye drop containing BAC as a preservative is applied after cataract surgery, it can produce irritation and BAC toxicity to the patient. To overcome this side effect the inventors of the present invention prepared BAC-free NSAID unit dose medication for above indication for better patient compliance. Further the elimination of BAC may be helpful in improving the stability of bromfenac.

Patients experiencing hypersensitivity reactions with benzalkonium chloride cannot use the commercial bromfenac product containing benzalkonium chloride as a preservative such as PROLENSA™ which is preserved with 0.005% w/v benzalkonium chloride. There is a necessity to prepare a stable, aqueous solution for eye ailments with reduced toxicity due to the presence of antimicrobial preservatives.

U.S. Pat. No. 8,129,431, US2012115957, US2013090384, US2007287749 and W02013055856 disclose an aqueous liquid preparation containing 2-amino-3-(4-bromobenzoyl)phenylacetic acid or its pharmacologically acceptable salt or a hydrate thereof, an alkyl aryl polyether alcohol type polymer such as tyloxapol, or a polyethylene glycol fatty acid ester such as polyethylene glycol monostearate and a preservative such as BAC.

The inventors of the present invention are preparing a stable, aqueous solution comprising bromfenac or a pharmacologically acceptable salt, polymorph, ester or hydrate thereof and/or pharmaceutically acceptable excipients wherein the invention is devoid of an alkyl aryl polyether alcohol type polymer such as tyloxapol, a polyethylene glycol fatty acid ester such as polyethylene glycol monostearate and BAC.

The stable, aqueous solution of the present invention is also devoid of antioxidants such as sulphite(s) but not limited to sodium sulfite, potassium sulphite and the like. Sodium sulfite is irritating to the eyes. Symptoms of irritation may include redness, itching or tearing as disclosed and mentioned in various companies Material Safety Data Sheet (MSDS) such as Santa Cruz Biotechnology, Inc.; LabChem, Inc., New Jersey department of health and senior services and the like.

The inventors of the present invention with expenditure of intellectual effort and careful experimentation have prepared an antioxidant-free stable, aqueous solution comprising a non-steroidal anti-inflammatory drug (NSAID) and/or pharmaceutically acceptable excipients for the treatment of ocular inflammation and pain after cataract surgery wherein more specifically, the present invention is preferably devoid of BAC.

Hence there was an unmet medical need to prepare a stable, aqueous solution comprising bromfenac or a pharmacologically acceptable salt, polymorph, ester or hydrate thereof and/or pharmaceutically acceptable excipients wherein the invention is devoid of an alkyl aryl polyether alcohol type polymer such as tyloxapol, a polyethylene glycol fatty acid ester such as polyethylene glycol monostearate and BAC.

There also is a need for a method for treating ocular inflammation and pain after cataract surgery wherein the method comprises a once a day topical application to the eye of the patient in need of a stable, aqueous solution comprising bromfenac or a pharmacologically acceptable salt, polymorph, ester or a hydrate thereof and/or pharmaceutically acceptable excipients.

### SUMMARY OF THE INVENTION

Preferably, the invention includes an aqueous pharmaceutical solution for treatment of ocular pain or inflammation comprising an initial amount of 0.1-1.0 mg/ml bromfenac (based on weight of free acid); wherein the solution is stable when stored for 6 months at 40° C. at no more than 40% relative humidity.

Preferably, the invention comprises a method of preparing a stable bromfenac solution comprising preparing an aqueous mixture comprising: an initial amount of 0.1-1.0 mg/ml bromfenac (based on weight of free acid); 0.5-4.0 mg/ml buffering agent; 0.2-1.5 mg/ml chelating agent; 2-40 mg/ml tonicity agent; and a pH adjusting agent in an effective amount such that the composition has a pH of 7.0-8.0.

Preferably, the invention comprises a method of preparing a stable bromfenac solution comprising preparing an aqueous mixture comprising: an initial amount of 0.1-1.0 mg/ml bromfenac (based on weight of free acid); 0.01-1.0 mg/ml surfactant; 0.5-4.0 mg/ml buffering agent; 0.2-1.5 mg/ml chelating agent; 2-40 mg/ml tonicity agent; and a pH adjusting agent in an effective amount such that the composition has a pH of 7.0-8.0.

Preferably, the solution comprises no benzalkonium chloride. Preferably, the solution comprises no organic antimicrobial compound.

Preferably, the solution, upon storage for 6 months at 40° C. at no more than 40% relative humidity, comprises a final amount of bromfenac (based on weight of free acid) greater than or equal to 97% of the initial amount. Preferably, the solution, upon storage for 6 months at 40° C. at no more than 40% relative humidity, comprises less than 1.2 w/v % of 7-(4-bromobenzoyl)-1,3-dihydro-2H-indol-2-one.

Preferably, the solution, upon storage for 4 weeks at 50° C. comprises a final amount of bromfenac (based on weight of free acid) greater than or equal to 97% of the initial amount. Preferably, the solution, upon storage for 4 weeks at 50° C. comprises less than 1.2 w/v % of 7-(4-bromobenzoyl)-1,3-dihydro-2H-indol-2-one.

Preferably, the solution does not comprise an alkyl aryl polyether alcohol type polymer or a polyethylene glycol fatty acid ester.

Preferably, the solution does not comprise a sulfite anti-oxidant.

Preferably, the solution further comprises a buffer. Preferably, the buffer comprises a citrate buffer. Preferably, the buffer does not comprise a borate buffer.

Preferably, the solution has a pH of 7 to 8.

Preferably, the solution is packaged in a unit dose container. Preferably, the solution is packaged in a unit dose kit form.

Preferably, the solution comprises 0.1-1.0 mg/ml bromfenac (based on weight of free acid); 0.5-4.0 mg/ml buffering agent; 0.2-1.5 mg/ml chelating agent; 2-40 mg/ml tonicity agent; and a pH adjusting agent in an effective amount such that the composition has a pH of 7-8.

Preferably, the solution comprises 0.1-1.0 mg/ml bromfenac (based on weight of free acid); 0.01-1.0 mg/ml surfactant; 0.5-4.0 mg/ml buffering agent; 0.2-1.5 mg/ml chelating agent; 2-40 mg/ml tonicity agent; and a pH adjusting agent in an effective amount such that the composition has a pH of 7-8.

Preferably, the solution has an osmolality of 250-350 mOsm/kg.

Preferably, the invention includes a method of treating ocular inflammation and/or pain, comprising applying at least once a day to an eye of a patient in need thereof the inventive bromfenac solution. The applying may be preferably done once or twice a day.

In one embodiment of the present invention, is to develop a stable, aqueous solution comprising a non-steroidal anti-inflammatory drug (NSAID) for the treatment of ocular inflammation and pain after cataract surgery.

In another embodiment of the present invention, is to prepare a stable, aqueous solution comprising bromfenac or a pharmacologically acceptable salt, polymorph, ester or hydrate thereof and/or pharmaceutically acceptable excipients wherein the invention is devoid of an alkyl aryl polyether alcohol type polymer such as tyloxapol, a polyethylene glycol fatty acid ester such as polyethylene glycol monostearate and BAC. As used in the present disclosure, the term “devoid” means that the formulation does not require the component in question for stability and/or efficacy. Preferably, the term “devoid” means that the formulation does not comprise, or comprises no more than a trace amount of, the component in question.

In yet another embodiment of the present invention, the stable, aqueous solution of the present invention is also devoid of antioxidants, preferably devoid of sulfites, more preferably devoid of sodium sulfite, potassium sulfite and the like.

Further in one embodiment of the present invention, citrate buffer is used instead of borate buffer.

Further in another embodiment of the present invention, is to provide a less irritant aqueous solution without BAC as a preservative which leads to safe, tolerable and patient compliant formulation while maintaining and/or improving its efficacy for the treatment of ocular inflammation and pain after cataract surgery.

Further in yet another embodiment of the present invention is to prepare a stable, aqueous solution wherein the formulation is devoid of any kind of antioxidants such as sulfite(s) which causes irritation to the eyes wherein symptoms of irritation may include redness, itching or tearing. So, the absence of Sodium sulfite adds to the benefit for better patient compliance.

In another embodiment, an aqueous composition comprising not more than about 0.005w/v % and preferably not more than about 0.003w/v % of benzalkonium chloride is provided wherein the composition contains nil or trace amounts of preservative such as benzalkonium chloride (BAC).

Furthermore in one embodiment of the present invention, is to provide a method for treating ocular inflammation and pain after cataract surgery wherein the method comprises application to the eye of the patient in need of a stable, aqueous solution comprising bromfenac or a pharmacologically acceptable salt, polymorph, ester or hydrate thereof and/or pharmaceutically acceptable excipients. Application is preferably once a day, but may be more than once per day, e.g., two times a day.

Furthermore in another embodiment, the present invention is to provide a method of using the inventive compositions.

Furthermore in yet another embodiment, the present invention is to provide a process of preparing a stable, aqueous solution comprising bromfenac or a pharmacologically acceptable salt, polymorph, ester or hydrate thereof and/or pharmaceutically acceptable excipients wherein the invention is devoid of an alkyl aryl polyether alcohol type polymer such as tyloxapol, a polyethylene glycol fatty acid ester such as polyethylene glycol monostearate and BAC.

Furthermore in yet another embodiment, the present bromfenac ophthalmic aqueous solution without preservative and antioxidants is a clear, isotonic, sterile solution and is useful for the treatment of ocular inflammation and pain after cataract surgery wherein the solution is contained in a unit dose kit form and is applied once a day to each eye.

### DETAILED DESCRIPTION OF THE INVENTION

As used herein, an “antioxidant” is a substance that when present in a mixture containing an oxidizable substrate biological molecule significantly delays or prevents oxidation of the substrate biological molecule. In the context of this invention, antioxidants include, but are not limited to, antioxidants that are irritating to the eyes. Such antioxidants include sodium sulfite, potassium sulphite, sodium metabisulfite, sodium thiosulfate, acetylcysteine, butylated hydroxyanisole, butylated hydroxytoluene and the like, and mixtures thereof.

As used herein, the term “Deg. C.” wherever appears is an abbreviation for “Degree Celsius”.

As used herein, the “Control A” wherever appears is 0.09% bromfenac solution, approved by the U.S. FDA, and believed to be qualitatively and quantitatively the same as XIBROM®.

As used herein, the “Control B” wherever it appears is the U.S. FDA approved 0.07% bromfenac product PROLENSA®.

The present invention is directed to stable, aqueous solution comprising a non-steroidal anti-inflammatory drug (NSAID) for the treatment of ocular inflammation and pain after cataract surgery.

The present invention is directed to prepare a stable, aqueous solution comprising bromfenac or a pharmacologically acceptable salt, polymorph, ester or hydrate thereof and/or pharmaceutically acceptable excipients wherein the invention is devoid of a alkyl aryl polyether alcohol type polymer such as tyloxapol, a polyethylene glycol fatty acid ester such as polyethylene glycol monostearate and/or BAC.

The present invention is to provide a method for treating ocular inflammation and pain after cataract surgery wherein the method comprises a once a day topical application to the eye of the patient in need of a stable, aqueous solution comprising bromfenac or a pharmacologically acceptable salt, polymorph, ester or hydrate thereof and/or pharmaceutically acceptable excipients.

The present invention is to provide a process of preparing a stable, aqueous solution comprising bromfenac devoid of an alkyl arylpolyether alcohol type polymer such as tyloxapol, a polyethylene glycol fatty acid ester such as polyethylene glycol monostearate and BAC.

In one of the embodiments, the preservative if used in the present invention is selected from the group consisted of, but not limited to benzethonium chloride, benzododecinium bromide, quaternary ammonium compounds such as but not limited to benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide and the like; Polyquaternium-1 (Polyquad®), 1phenyl ethanol, phenyl propanol, phenyl mercuric acetate, phenyl mercuric nitrate, phenyl mercuric borate, chlorhexidine acetate or gluconate, chlorocresol, benzoic acid, benzyl alcohol, butylparaben, propylparaben, methylparaben, chlorobutanol, phenoxyethanol, sodium methyl paraben, sodiumpropyl paraben, thimerosal, and mixtures thereof, wherein the said preservatives may be used in an amount from 0.001% to 0.5%; or from 0.005% to 0.5%.

In another embodiment, an aqueous composition comprising not more than about 0.005w/v % and preferably not more than about 0.003w/v % of benzalkonium chloride is provided wherein the composition contains nil or a trace amounts of preservative such as benzalkonium chloride (BAC).

The present bromfenac ophthalmic aqueous solution without preservative such as BAC and antioxidants such as sodium sulfite, potassium sulphite is a clear, isotonic, sterile solution and is useful for the treatment of ocular inflammation and pain after cataract surgery wherein the solution may be contained in a multi dose kit or in a unit dose kit and is applied once a day to each eye.

In another embodiment, aqueous solution of the present invention may also be packaged in a single-use container.

In yet another embodiment, aqueous solution of the present invention may also be packaged in a multi-use container.

In another embodiment the stable, ophthalmic compositions can also be prepared as one of the embodiments of the present invention to make the composition pharmaceutically acceptable for used as a single unit dose to avoid or reduce ocular toxicity experiencing hypersensitivity reactions to the patients by BAC used to preserve the ophthalmic preparations.

Any concentration of NSAID that is effective can be used, and can be determined by one of ordinary skill in the art using the present disclosure as guidance. When the NSAID comprises bromfenac, bromfenac is preferably present in a concentration of 0.1-1.0 mg/ml (based on bromfenac free base). Some preferred concentrations include 0.2, 0.4, 0.5, 0.6, 0.7, 0.8, and 0.9 mg/ml bromfenac and ranges formed from these values. These bromfenac concentrations may be used with the excipients and amounts thereof listed in Table 1.

The aqueous solution of the present invention is stable at 50 Deg. C. for four (4) weeks wherein the stability result clearly demonstrates that the assay of bromfenac and related substance are well within specification ranges. The bromfenac content in the formulation of present invention is 99.5%, compared to 99.6% of the label in “Control A” formulation at 50Deg. C. for four (4) weeks. Also the related substance in the formulation of present invention is only 0.46%, compared to 0.28% in the “Control A” formulation at 50Deg. C. for four (4) weeks. These differences in content of bromfenac and related substances are within the margin of error of analysis. Hence, one can conclude that the formulation of present invention is unexpectedly as stable as that of the “Control A” in a neutral pH range, without the need for, or use of, BAC and/or antioxidants such as sulfite(s) such as sodium sulfite, potassium sulfite, and the like, and/or alkyl aryl polyether alcohol type polymer or polyethylene glycol fatty acid ester.

“Stable” refers to low bromfenac degradation and/or low formation of degradation products (or “related substance”) after aging. Stable formulations preferably include those in which bromfenac assay is greater than or equal to 97%, more preferably 98%, 99% or 99.5% after aging. Stable formulations preferably include those in which the related substance assay is less than 1.2%, more preferably less than 1.0, 0.7, 0.5% after aging. Aging may be accelerated or non-accelerated, preferably accelerated. Accelerated aging preferably comprises storage at 50° C. for four (4) weeks, preferably in a closed container in the dark. As is known in the art, “related substances” preferably includes regulated by regulatory authorities, e.g., the U.S. FDA. Related substances preferably include Impurity A as well as the highest unknown impurity, and more preferably includes all bromfenac degradation products. These impurities can be determined and assayed by one of ordinary skill in the art. As is known in the art, Impurity A is 7-(4-bromobenzoyl)-1,3-dihydro-2H-indol-2-one.

In one of the preferred embodiments, excipients used are ophthalmically acceptable which includes, without limited to, buffering agents, chelating agents, tonicity agents, permeation enhancers, surfactants, pH adjusting agents and the like.

In one of the embodiments of the present invention, buffering agents used in the present invention includes but are not limited to acetate buffers, citrate buffers, phosphate buffers, sodium dihydrogen phosphate dihydrate, dibasic sodium phosphate heptahydrate, monobasic sodium phosphate, citric acid, citric acid monohydrate or ε-aminocaproic acid and the like.

In another embodiments, the present invention may include chelating agents but not limited to disodium edetate or ethylenediamine tetraacetic acid (“EDTA”), diammonium EDTA, dipotassium EDTA, calcium disodium EDTA, hydroxyethylethylenediaminetriacetic acid (“HEDTA”), ethylenediaminetetraacetic acid, mono(triethanolamine) salt (“TEA-EDTA”), tetrasodium EDTA, tripotassium EDTA, trisodium phosphate, diammonium citrate, galactaric acid, galacturonic acid, gluconic acid, glucuronic acid, cyclodextrin, potassium citrate, the potassium salt of ethylenediamine-tetra (methylene phosphonic acid) (“EDTMP”), sodium citrate, sodium EDTMP, and the like.

In yet another embodiment of the present invention, tonicity adjusting agents may be added and included without limitation such as glycerin, sorbitol, sodium hydroxide, sodium chloride, potassium chloride, and mannitol, dextrose, propylene glycol and combinations thereof or any other suitable ophthalmically acceptable tonicity adjusting agents.

In one of the preferred embodiments of the present invention, vehicles can also be used in the ophthalmic compositions of the present embodiments. These vehicles include, but are not limited to, methyl cellulose, hydroxypropyl methyl cellulose, poloxamers, carboxymethyl cellulose, hydroxyethyl cellulose, poly ethylene glycol, hyaluronic acid, polygalacturonic acid, xyloglucan, carbopol, polycarbophil, gellan gum physiological saline solution, water, purified water, and combinations thereof.

The present invention is devoid of any kind of antioxidants such as sodium sulfite, potassium sulpfite, sodium metabisulfite, sodium thiosulfate, acetylcysteine, butylated hydroxyanisole, butylated hydroxytoluene and the like and mixtures thereof which causes irritation to the eyes.

In another embodiment, the surfactants if used may be selected from the group consisted of, but are not limited to sodium lauryl sulfate, docusate sodium, polyoxyalkyl ethers, polyoxylalkyl phenyl ethers, polyoxyl 40 hydrogenated castor oil (Cremophor RH 40), polyoxy hydrogenated castor oil, polyoxy sorbitan esters, sorbitan esters, polysorbates, polyoxyl 35 castor oil, sorbitan monolaureates, poloxamer and mixtures thereof.

The pH adjusting agents include hydrochloric acid, sodium hydroxide, phosphoric acid, acetic acid and the like.

The excipients used in the present invention are preferably selected to be non-toxic and have no substantial detrimental effect (preferably, in the amount used) on the present ophthalmic compositions, on the use of the compositions or on the human or animal to which the ophthalmic compositions are to be administered.

In one of the preferred embodiments, the present invention provide the ophthalmic compositions in the form of aqueous liquids, solutions, emulsion, dispersion, suspension, reverse emulsion and microemulsion, nanoemulsion, nano reservoir system, in-situ gel drops, nanoparticulate system, liposomal drops, bioadhesive gel drops, drops and the like.

In another embodiment, the present invention preferably provides the ophthalmic compositions for topical ophthalmic delivery comprising administering said composition in the eyes, ear, and/or nose of the humans or animals.

In a preferred embodiment, the stable, solution would be an aqueous solution having a pH value within the range of from about 6.5 to about 8, especially from about 7.0 to about 8.0 and preferably from about 7.2 to about 7.8 and osmolality in range of at least about 250 mOsmol/kg and/or less than or equal to about 350 mOsmol/kg.

In an especially useful embodiment, the osmolality or tonicity of the carrier component substantially corresponds to the tonicity of the fluids of the eye, in particular the human eye. The pH of the aqueous solution of the present invention is closer to ocular or lacrimal fluid as compared to the marketed product.

In a yet further preferred embodiment, the present invention provides a process of preparing a stable, aqueous solution comprising bromfenac and/or pharmaceutically acceptable excipients.

Still further, the present invention may also be presented as a kit comprising a stable, stable, aqueous solution comprising bromfenac and/or pharmaceutically acceptable excipients, the aqueous solution being contained within a container prepared from a pharmaceutically acceptable packaging material.

Any pharmaceutically acceptable packaging material may be used, preferably packaging material that is suitable for containing ophthalmic aqueous solution, more preferably bromfenac ophthalmic aqueous solution. Pharmaceutically acceptable packaging materials include but are not limited to low density polyethylene (“LDPE”), high density polyethylene (“HDPE”), polypropylene, polystyrene, polycarbonate, polyesters (such as polyethylene terephthalate and polyethylene naphthalate), nylon, polyvinyl chloride), poly(vinylidine chloride), poly(tetrafluoroethylene) and other materials known to those of ordinary skill in the art. Flexible bottles prepared from, or comprising, LDPE, HDPE or polypropylene are particularly preferred.

Preferred containers include bottles, preferably a dropper (e.g., a bottle or ampule suitable for dropwise application of the composition), more preferably, a single-use bottle or dropper. The containers are preferably sterilized, preferably prior to filling. Any suitable method can be used to sterilize the containers, and can be determined by the person of ordinary skill in the art. Some preferred methods include exposure to gamma irradiation and/or exposure to ethylene oxide gas.

The aqueous solution is preferably sterile. An article comprising the aqueous solution filled in a container is preferably sterile, preferably at the time the container is filled. The aqueous solution is preferably filled into sterile multi-use or single-use containers, preferably single-use containers.

The present invention provides a method for treating ocular inflammation and pain after cataract surgery wherein the method comprises a once a day topical application to the eye of the patient in need of an antioxidants-free stable, aqueous solution comprising bromfenac or a pharmacologically acceptable salt, polymorph, ester or a hydrate thereof and/or pharmaceutically acceptable excipients wherein more specifically, the present invention is preferably devoid of BAC.

The present invention provides a method of using the inventive compositions for treating ocular inflammation and pain after cataract surgery.

The present invention provides a process of preparing an antioxidants-free stable, aqueous solution comprising bromfenac or a pharmacologically acceptable salt, polymorph, ester or a hydrate thereof and/or pharmaceutically acceptable excipients wherein more specifically, the present invention is preferably devoid of BAC.

In a preferred embodiment, the present invention provides a process of preparing antioxidants free stable, aqueous solution wherein the composition is prepared by a process comprising, preferably with continuous nitrogen purging:

1. Add quantity of purified water approximately 80% of the batch size in a container, e.g., a stainless steel vessel.

2. Add one component of a buffer system (e.g., Sodium citrate dihyrate) to step 1 under stirring and mix until dissolved.

3. Add chelating agent (e.g., disodium edetate) to step 2 under stirring and mix until dissolved.

4. Add mannitol to step 3 under stirring and mix until dissolved.

5. Add NSAID (e.g., bromfenac) to step 4 under stirring and mix until dissolved.

6. Check pH of the solution; adjust if necessary to pH 7.0-8.0.

7. Make up the volume to 100% of the batch size with purified water and stir for 5 minutes.

As can be determined by one of skill in the art using the present disclosure as a guide, the order of the above steps can be changed, and two or more steps may be combined.

### Stability Studies:

An antioxidants-free stable, aqueous solution of Bromfenac are prepared with ranges of ingredients as shown in Table 1, and exposed to stress studies at 50 Deg. C. for 0 day; 15 days and 30 days to determine the stability of the proposed formulations. A comparative study is initiated to demonstrate the stability of present invention formulation with a generic version which is 0.09% bromfenac solution, approved by the U.S. FDA, and believed to be qualitatively and quantitatively the same as XIBROM® (herein defined as “Control A”) as shown in Table 2. The initial pH of the “Control A” is 8.2, and of present invention is 7.7.

A non-accelerated or accelerated study method may be used to test stability. A preferred stress (or accelerated) study comprises placing the composition/solution is filled in Opaque LDPE vial with LDPE nozzle and HDPE cap, packing in secondary packaging material, and maintaining at 50 Deg. C. in the dark. Impurities are measured by HPLC for initial (0 days), 15 days and 30 days.

As understood by those of skill in the art, when the NSAID comprises bromfenac, the impurities preferably measured include Impurity A (7-(4-Bromobenzoyl)-1,3-dihydro-2H-indol-2-one), and total impurities, as well as identification of the amount of the highest unknown impurity.

TABLE 1

| Ingredients | Ranges varied in mg/ml |
|---|---|
| Bromfenac | Content of Bromfenac sodium |
| | hydrate is kept at 1.035 mg/ml |
| | (eq. to 0.9 mg/ml of Bromfenac). |
| Edetate Disodium | 0.2-1.5 |
| Sodium citrate dihydrate | 0.5-4.0 |
| Mannitol | 2-40 |
| Sodium chloride | 0.5-6.0 |
| Sodium hydroxide | QS |
| MilliQ water | QS |
| pH | 7.0-8.0 |

TABLE 2
Comparative study

| Formulation composition | “Control A” | Present Invention (Example 1) |
|---|---|---|
| Ingredients | mg/ml | mg/ml |
| Bromfenac sodium hydrate | 1.035 | 1.035 |
| Boric acid | 11 | X |
| Edetate disodium | 0.2 | 1 |
| Polysorbate 80 | 1.5 | X |
| Sodium borate | 11 | X |
| Sodium sulfite anhydrous | 2 | X |
| Benzalkonium chloride | 0.05 | X |
| Sodium citrate dihydrate | X | 2.92 |
| Mannitol | X | 20 |
| Sodium chloride | X | 4.5 |
| Sodium hydroxide | QS | QS |
| MilliQ water | QS | QS |

### Results and Observations:

The controlled formulation (“Control A”) with BAC is evaluated for bromfenac content and related substances at initial; at two (2) weeks and at four (4) weeks for stress stability at 50 Deg. C. in the dark. Results are shown in Table 3.

The formulation of present invention is evaluated for bromfenac content and related substances at initial; at two (2) weeks and at four (4) weeks for stress stability at 50 Deg. C. in the dark. Results are shown in Table 4.

TABLE 3

| | Specs. | Initial, | 2 Weeks/50° C. | 4 Weeks/50° C. |
|---|---|---|---|---|
| Parameters | Limits | “Control A” | “Control A” | “Control A” |
| Description | | Clear greenish | Not performed | Clear greenish |
| | | yellow coloured | | yellow coloured |
| | | solution | | solution |
| pH | 7.9-8.7 | 8.24 | 8.34 | 8.27 |
| Impurity A | 1% | Not detected | Not detected | Not detected |
| Highest unknown | 1% | 0.05% | 0.05% | 0.17% |
| impurity | | | | |
| Total impurities | 3% | 0.05% | 0.05% | 0.28% |
| Assay | 90-110% | 99.7% | 99.1% | 99.6% |

TABLE 4

| | Specs. | | | |
|---|---|---|---|---|
| Parameters | Limits | Initial | 2 Weeks/50° C. | 4 Weeks/° C. |
| Description | | Clear greenish | Clear greenish | Clear greenish |
| | | yellow coloured | yellow coloured | yellow coloured |
| | | solution | solution | solution |
| pH | 7.0-8.0 | 7.7 | 7.2 | 7.2 |
| Impurity A | 1% | Not detected | 0.06% | 0.08% |
| Highest unknown | 1% | 0.05% | 0.07% | 0.12% |
| impurity | | | | |
| Total impurities | 3% | 0.05% | 0.18% | 0.46% |
| Assay | 90-110% | 99.2% | 100.3% | 99.5% |
| Content of | NA | NA | NA | NA |
| sodium sulfite | | | | |

### EXAMPLES

The scope of the present invention is illustrated by the following examples which are not meant to restrict the scope of the invention in any manner whatsoever.

The term ‘q.s.’ wherever appears in the examples is an abbreviation for ‘quantity sufficient’ which is the amount of the excipient in such quantities that is just sufficient for its use in the composition of the present invention.

The term ‘Deg. C.’ wherever appears is an abbreviation for “Degree Celsius” and the term “NMT” wherever appears is an abbreviation for “Not More Than”.

Example I exhibits good stability following the stress stability test at 50 Deg. C. for four (4) weeks (e.g., 30 days).

### Example 1

TABLE 5

| Ingredients | Quantity |
|---|---|
| (Formula 1) | (mg/ml) |
| Bromfenac sodium hydrate | 1.035 |
| Edetate Disodium | 1 |
| Sodium citrate dihydrate | 2.92 |
| Mannitol | 20 |
| Sodium chloride | 4.5 |
| Sodium hydroxide | QS |
| MilliQ water | QS |

### Method:

1. Add quantity of purified water approximately 80% of the batch size in a chosen stainless steel vessel.

2. Add sodium citrate dihyrate to step 1 under stirring and mix until dissolved.

3. Add edetate disodium to step 2 under stiffing and mix until dissolved.

4. Add mannitol to step 3 under stirring and mix until dissolved.

5. Add bromfenac sodium hydrate to step 4 under stirring and mix until dissolved.

6. Continuous Nitrogen purging from step 2 to step 5

7. Check pH of the solution; adjust if necessary to pH 7.0-8.0.

8. Make up the volume to 100% of the batch size with purified water and stir for 5 minutes.

The Results of Stress Stability at 50 Deg C. for 4 Weeks for “Control A” with BAC:

The Bromfenac content is measured and found to be 99.6% (Limit: 90.0 -110.0%), the highest unknown impurity is measured and found to be 0.17% (Limit: NMT 1.0%) and total impurity is measured and found to be 0.28% (Limit: NMT 3.0%).

### The Results of Stress Stability at 50 Deg C. for 4 Weeks for Present Invention (Example 1):

The Bromfenac content is measured to be 99.5% (Limit: 90.0 -110.0%), the highest unknown impurity is measured to be 0.12% (Limit: NMT 1.0%) and total impurity is measured to be 0.46% (Limit: NMT 3.0%).

The conducted comparative Stability studies between “Control A” (with BAC and antioxidants) and present invention (Example 1) (without BAC and antioxidants) shows that the ingredients used in the formulations have varied effects on the stability of the formulations investigated when measured at different time periods (at initial; at two (2) weeks and at four (4) weeks) at 50 Deg. C.

Formulations of the present invention are unexpectedly as stable as that of the “Control A” in a neutral pH range, without the need for, or use of, BAC and/or antioxidants such as sulfite(s) such as sodium sulfite, potassium sulfite, and the like, and/or alkyl aryl polyether alcohol type polymer or polyethylene glycol fatty acid ester.

The study for Example 1 is further extended to two more stability conditions:

(a) 6-months accelerated study with storage under conditions of 40° C. and 25% relative humidity. Results for 6-months accelerated testing are provided in Table 9 (Formulation 1) and Table 10 (“Control A”).

(b) A 6-month long term study is also performed, with storage under conditions of 25° C. and 40% relative humidity. Results are shown in Table 11.

### Example 2

One more formulation is also prepared according to Table 6, having concentration of 0.07 wt/vol % based on weight of bromfenac free base:

TABLE 6

| Ingredients | Quantity |
|---|---|
| Formula 2 | (mg/ml) |
| Bromfenac sodium hydrate | 0.805 |
| Disodium edetate | 1.00 |
| Polysorbate 80 | 0.10 |
| Sodium citrate dihydrate | 2.92 |
| Mannitol | 20.00 |
| Sodium chloride | 4.50 |
| NaOH/HCl | q.s. |
| Water for Injection | q.s. to 1 ml |

The above formulation (Formula 2), is tested for stability against a control formulation (herein referred to as “Control B”). Six-month accelerated testing refers to storage at 40° C., at not more than 25% relative humidity. Results for 6-months accelerated testing are provided in Table 7 (“Control B”, comprising 0.005 w/v % BAC), Table 8 (0.07% formulation of Table 6 (Formula 2); first 2 months).

Also four weeks stress testing is also performed at 50° C. Table 12 provides four weeks stress results for the 0.07% formulation of Table 6 (Formula 2), and Table 13 provides four weeks stress results for reference “Control B”.

TABLE 7

| | | Schedule Period | | | | |
|---|---|---|---|---|---|---|
| | Specification | Initial | 1 Month | 2 Month | 3 Month | 6 Month |
| Test Parameter | | | | | | |
| Appearance | Not Applicable | Clear, | Clear, | Clear, | Clear, greenish | Clear, greenish |
| | | greenish | greenish | greenish | yellow | yellow solution. |
| | | yellow | yellow | yellow | solution. | |
| | | solution. | solution. | solution. | | |
| Assay (By HPLC) | Not Applicable | 99.1% | NA | NA | 97.0% | 92.7% |
| Bromfenac free acid- | | | | | | |
| 0.9 mg | | | | | | |
| Osmolality (mOsmol/kg) | Not Applicable | 310 | NA | NA | 320 | 335 |
| pH | Not Applicable | 7.78 | NA | NA | 7.80 | 7.78 |
| Related substances (By | | | | | | |
| HPLC) | | | | | | |
| Bromfenac sodium | Not Applicable | 0.02% | Not | Not Detected | Not Detected | Below |
| impurity A | | | Detected | | | Quantification |
| | | | | | | Limit |
| Highest unknown | Not Applicable | Below | 0.06% | 0.25% | 0.49% | 1.43% |
| impurity | | disregard | | | | |
| | | limit | | | | |
| Total Impurities | Not Applicable | 0.02% | 0.06% | 0.40% | 0.77% | 2.83% |

TABLE 8

| | | Schedule Period | | |
|---|---|---|---|---|
| Test Parameter | Specification | Initial | 1 Month | 2 Month |
| Appearance | Clear, green- | Clear, green- | Clear, green- | Clear, green- |
| | ish yellow | ish yellow | ish yellow | ish yellow |
| | solution. | solution. | solution. | solution. |
| Assay (By HPLC) | Between 90.0% and | 99.0% | 100.0% | 102.1% |
| Bromfenac free acid- | 110.0% of labeled | | | |
| 0.9 mg | amount of bromfenac | | | |
| | free acid. | | | |
| Osmolality | 250-350 | 298 | 303 | 306 |
| (mOsmol/kg) | mOsmol/kg | | | |
| pH | 7.0-8.0 | 7.66 | 7.58 | 7.47 |
| Related substances | | | | |
| (By HPLC) | | | | |
| Bromfenac sodium | Not more than | 0.01% | 0.10% | 0.14% |
| impurity A | 1.0% | | | |
| Highest unknown | Not more than | 0.08% | 0.10% | Below dis- |
| impurity | 1.0% | | | regard limit |
| Total Impurities | Not more than | 0.09% | 0.20% | 0.22% |
| | 3.0% | | | |

TABLE 9

| | | Schedule Period | | | | |
|---|---|---|---|---|---|---|
| | Specification | Initial | 1 Month | 2 Months | 3 Months | 6 Months |
| Test Parameter | | | | | | |
| Appearance | Clear, greenish | Clear, | Clear, | Clear, | Clear, greenish | Clear, greenish |
| | yellow solution. | greenish | greenish | greenish | yellow | yellow solution. |
| | | yellow | yellow | yellow | solution. | |
| | | solution. | solution. | solution. | | |
| Assay (By HPLC) | Between | 98.50% | 100.50% | 99.60% | 99.90% | 99.80% |
| Bromfenac free acid-0.9 mg | 90.0% and | | | | | |
| | 110.0% of | | | | | |
| | labeled amount | | | | | |
| | of bromfenac | | | | | |
| | free acid. | | | | | |
| Osmolality (mOsmol/kg) | 250-350 | 304 | 305 | 308 | 311 | 320 |
| | mOsmol/kg | | | | | |
| pH | 7.0-8.0 | 7.49 | 7.39 | 7.42 | 7.41 | 7.44 |
| Related substances (By | | | | | | |
| HPLC) | | | | | | |
| Bromfenac sodium | Not more than | 0.03% | 0.06% | 0.07% | 0.10% | 0.14% |
| impurity A | 1.0% | | | | | |
| Highest unknown | Not more than | 0.08% | 0.09% | 0.10% | 0.11% | 0.08% |
| impurity | 1.0% | | | | | |
| Total Impurities | Not more than | 0.19% | 0.24% | 0.26% | 0.30% | 0.37% |
| | 3.0% | | | | | |

TABLE 10

| | | Schedule Period | | | | |
|---|---|---|---|---|---|---|
| | Specification | Initial | 1 Months | 2 Months | 3 Months | 6 Months |
| Test Parameter | | | | | | |
| Appearance | Clear, greenish | Clear, | Clear, | Clear, | Clear, greenish | Clear, greenish |
| | yellow solution. | greenish | greenish | greenish | yellow | yellow solution. |
| | | yellow | yellow | yellow | solution. | |
| | | solution. | solution. | solution. | | |
| Assay (By HPLC) | Between | 99.60% | 98.90% | 97.60% | 97.30% | 97.10% |
| Bromfenac free acid-0.9 mg | 90.0% and | | | | | |
| | 110.0% of | | | | | |
| | labeled amount | | | | | |
| | of bromfenac | | | | | |
| | free acid. | | | | | |
| Osmolality (mOsmol/kg) | 250-350 | 296 | 304 | 307 | 317 | 327 |
| | mOsmol/kg | | | | | |
| pH | 7.8-9.0 | 8.50 | 8.41 | 8.36 | 8.31 | 8.32 |
| Related substances (By | | | | | | |
| HPLC) | | | | | | |
| Bromfenac sodium | Not more than | ND | ND | ND | ND | ND |
| impurity A | 1.0% | | | | | |
| Highest unknown | Not more than | ND | ND | 0.05% | 0.18% | 0.70% |
| impurity | 1.0% | | | | | |
| Total Impurities | Not more than | ND | ND | 0.10% | 0.40% | 1.44% |
| | 3.5% | | | | | |

TABLE 11

| | Schedule Period | | |
|---|---|---|---|
| Test Parameter | Initial | 3 Months | 6 Months |
| Appearance | Clear, green- | Clear, green- | Clear, green- |
| | ish yellow | ish yellow | ish yellow |
| | solution. | solution. | solution. |
| Assay (By HPLC) | 98.5 | 99.1 | 98.0 |
| Bromfenac | | | |
| free acid- | | | |
| 0.9 mg | | | |
| Osmolality | 304 | 305 | 306 |
| (mOsmol/kg) | | | |
| pH | 7.49 | 7.43 | 7.42 |
| Related substances | | | |
| (By HPLC) | | | |
| Bromfenac sodium | 0.03 | 0.07 | 0.09 |
| impurity A | | | |
| Highest unknown | 0.08 | 0.09 | 0.08 |
| impurity | | | |
| Total Impurities | 0.19 | 0.24 | 0.25 |

TABLE 12

| | Schedule Period | | |
|---|---|---|---|
| Test Parameter | Initial | 2 Weeks | 4 Weeks |
| Appearance | Clear, green- | Clear, green- | Clear, green- |
| | ish yellow | ish yellow | ish yellow |
| | solution. | solution. | solution. |
| Assay (By HPLC) | 99.0% | 100.9% | 102.0% |
| Bromfenac | | | |
| free acid- | | | |
| 0.9 mg | | | |
| pH | 7.66 | 7.54 | 7.57 |
| Related substances | | | |
| (By HPLC) | | | |
| Bromfenac sodium | 0.01% | 0.10% | 0.12% |
| impurity A | | | |
| Highest unknown impurity | 0.08% | 0.09% | 0.10% |
| Total Impurities | 0.09% | 0.19% | 0.28% |

TABLE 13

| | Schedule Period | | |
|---|---|---|---|
| Test Parameter | Initial | 2 Weeks | 4 Weeks |
| Appearance | Clear, green- | Clear, green- | Clear, green- |
| | ish yellow | ish yellow | ish yellow |
| | solution. | solution. | solution. |
| Assay (By HPLC) | 99.1% | NA | 99.30% |
| Bromfenac | | | |
| free acid- | | | |
| 0.9 mg | | | |
| pH | 7.78 | NA | 7.83 |
| Related substances | | | |
| (By HPLC) | | | |
| Bromfenac sodium | 0.02% | 0.01% | ND |
| impurity A | | | |
| Highest unknown | Below dis- | 0.14% | 0.16% |
| impurity | regard limit | | |
| Total Impurities | 0.02% | 0.15% | 0.33% |

1. An aqueous pharmaceutical solution for treatment of ocular pain or inflammation comprising an initial amount of 0.1-1.0 mg/ml bromfenac (based on weight of free acid); wherein the solution is stable when stored for 6 months at 40° C. at no more than 40% relative humidity; and wherein the solution does not comprise benzalkonium chloride.

2. The solution of claim 1, which upon storage for 6 months at 40° C. at no more than 40% relative humidity, comprises a final amount of bromfenac (based on weight of free acid) greater than or equal to 97% of the initial amount.

3. The solution of claim 1, which upon storage for 6 months at 40° C. at no more than 40% relative humidity, comprises less than 1.2 w/v% of 7-(4-bromobenzoyl)-1,3-dihydro-2H-indol-2-one.

4. The solution of claim 1, which does not comprise an alkyl aryl polyether alcohol type polymer or a polyethylene glycol fatty acid ester.

5. The solution of claim 1, which does not comprise an organic anti-microbial compound.

6. The solution of claim 1, which does not comprise a sulfite anti-oxidant.

7. The solution of claim 1, further comprising a buffer.

8. The solution of claim 7, wherein the buffer comprises a citrate buffer.

9. The solution of claim 1, which does not comprise a borate buffer.

10. The solution of claim 1, having a pH of 7 to 8.

11. The solution of claim 1, in a unit dose kit form, and having a pH of 7 to 8.

12. The solution of claim 1, comprising:
0.1-1.0 mg/ml bromfenac (based on weight of free acid);
0.5-4.0 mg/ml buffering agent;
0.2-1.5 mg/ml chelating agent;
2-40 mg/ml tonicity agent; and
a pH adjusting agent in an effective amount such that the solution has a pH of 7-8.

13. The solution of claim 1, comprising:
0.1-1.0 mg/ml bromfenac (based on weight of free acid);
0.01-1.0 mg/ml surfactant;
0.5-4.0 mg/ml buffering agent;
0.2-1.5 mg/ml chelating agent;
2-40 mg/ml tonicity agent; and
a pH adjusting agent in an effective amount such that the solution has a pH of 7-8.

14. The solution of claim 12, having an osmolality of 250-350 mOsm/kg.

15. The solution of claim 1, which is devoid of any preservative.

16. The solution of claim 1, wherein the solution is packaged in a unit dose container.

17. The solution of claim 14, which does not comprise a preservative, or which is packaged in a unit dose container.

18. A method of treating ocular inflammation and/or pain, comprising applying once a day to an eye of a patient in need thereof the solution of claim 1.

19. The method of claim 18, wherein the applying is done twice a day.

20. The method of claim 18, wherein the applying is done at least once a day.

21. A method of preparing a stable bromfenac solution comprising preparing an aqueous mixture comprising:
an initial amount of 0.1-1.0 mg/ml bromfenac (based on weight of free acid);
0.5-4.0 mg/ml buffering agent;
0.2-1.5 mg/ml chelating agent;
2-40 mg/ml tonicity agent; and
a pH adjusting agent in an effective amount such that the composition has a pH of 7-8;
wherein the bromfenac solution does not comprise benzalkonium chloride.

22. A method of preparing a stable bromfenac solution comprising preparing an aqueous mixture comprising:
an initial amount of 0.1-1.0 mg/ml bromfenac (based on weight of free acid);
0.01-1.0 mg/ml surfactant;
0.5-4.0 mg/ml buffering agent;
0.2-1.5 mg/ml chelating agent;
2-40 mg/ml tonicity agent; and
a pH adjusting agent in an effective amount such that the composition has a pH of 7-8;
wherein the bromfenac solution does not comprise benzalkonium chloride.

23. The method of claim 21, wherein the stable bromfenac solution does not comprise an organic antimicrobial compound.

24. The method of claim 21, wherein the bromfenac solution does not comprise an alkyl aryl polyether alcohol type polymer or a polyethylene glycol fatty acid ester.

25. The method of claim 21, further comprising packaging the aqueous mixture into a unit dose container.

26. The method of claim 21, wherein upon storage for 6 months at 40° C. at no more than 40% relative humidity, the bromfenac solution comprises a final amount of bromfenac (based on weight of free acid) greater than or equal to 97% of the initial amount.

27. The method of claim 21, wherein upon storage for 6 months at 40° C. at no more than 40% relative humidity, the bromfenac solution comprises less than 1.2 w/v % of 7-(4-bromobenzoyl)-1,3-dihydro-2H-indol-2-one.
